(19) United States
(12) Patent Application Publication
(10) Pub. No.: US 2024/0269487 A1
(43) Pub. Date: Aug. 15, 2024

(54) SYSTEM AND METHOD OF GAMMA RADIATION CONVERGENCE

(71) Applicants: Donald Medlin, Greenville, SC (US); Xiao Ran Zheng, Central, SC (US); Jad Saud, Debrecen (HU)

(72) Inventors: Donald Medlin, Greenville, SC (US); Xiao Ran Zheng, Central, SC (US); Jad Saud, Debrecen (HU)

(73) Assignee: Spinotron Kft, Debrecen (HU)

(21) Appl. No.: 18/441,560

(22) Filed: Feb. 14, 2024

Related U.S. Application Data
(60) Provisional application No. 63/485,209, filed on Feb. 15, 2023.

(51) Int. Cl. A61N 5/10

(52) U.S. Cl. CPC A61N 5/1082; A61N 2005/1087; A61N 2005/1089

(57) ABSTRACT

A system and method of gamma radiation convergence for the treatment of intracranial tumors. The system is comprised of a hexapod or six degree of freedom parallel robotic system to move a radiation head with respect to a rotating gantry.

### CROSS-REFERENCES TO RELATED APPLICATIONS

This application claims priority of U.S. Provisional Application No. 63/485,209, filed Feb. 15, 2023 incorporated by reference in its entirety.

### FIELD OF INVENTION

The subject invention relates to a system and method of converging gamma radiation in the treatment of intracranial tumors.

### BACKGROUND

Stereotactic Radiosurgery (SRS) is a minimally invasive treatment that delivers a large and typically single dose of radiation to a specific intracranial target while sparing surrounding healthy tissue. Its selective destruction is dependent mainly on sharply focused high-dose radiation that has a steep dose gradient away from the defined target. The biological effect is irreparable cellular damage and delayed vascular occlusion within the high-dose target volume. Since destructive doses are used, any normal structure in the target volume is subject to damage. There are several major devices that are used for SRS, however, they all use the same fundamental technique to deliver the total prescribed radiation dose to the target volume while reducing the damage to surrounding healthy tissue. The fundamental technique is to focus multiple nonparallel beams of external radiation on a stereotactically defined target. As any single beam passes through the patient, the radiation dose is deposited in the structures from the skin to the leading edge of the target volume (called entrance dose), within the target volume (target dose), and from the trailing edge of the target volume to the skin on the opposing side (exit dose). Each beam shares a common focal spot within the target volume, and thus the averaging of these intersecting beams results in a high dose of radiation to the target volume but innocuously low doses to surrounding structures. Furthermore, as the number of radiation beam trajectories through the surrounding healthy tissue increases, the radiation flux of each beam can be decreased, such that the total radiation energy remains the same, while the entrance and exit dose along each pathway is reduced. Stated another way: by increasing the accumulated solid angle of beam trajectories through the patient and reducing the radiation flux per trajectory, the prescribed dose will remain the same while reducing the damage to the surrounding healthy tissue. Thus, the accumulated solid angle of the beams directly affects the radiation conformity to the target and the spread of radiation outside of the targeted volume. For this reason, many technological advancements in modern SRS/SBRT resulted from attempts to increase solid angle coverage. Stereotactic Body Radiotherapy (SBRT) is the same fundamental technique as SRS but it is delivered to extracranial targets. Examples of prior art systems are described below.

Gamma Knife: The newest model of the Gamma Knife system utilizes 192 radioactive Cobalt-60 sources, that are distributed outside of a conical/hemispherical collimation system. A Gamma Knife System is shown in FIG. 2. It produces static nonparallel beams of collimated gamma radiation that converge to a common focal spot called the radiation isocenter, which is also static. The size of the radiation beams can be changed by toggling between various collimator apertures. With the beams turned on, the averaging of these beams at the isocenter produces a high-dose, quasi-spherical isocenter with a sharp dose gradient at its boundary. Each collimator aperture produces a spherical dose shot of different diameters, and the total dose deposited for each shot is directly proportional to the beam-on time. Since the isocenters are static, the location of the shot within the target volume is adjusted by moving the position of the patient. The prescribed dose to the target volume is achieved by packing one or multiple spherical shots into the target volume. Although the total accumulated solid angle is significant, it is also limited to less than 2 steradian coverage, because of the quasihemispherical distribution of static beams. It must be noted that the radiation beams have 0 degrees-of-freedom (DOF) motion.

Rotating Gamma Systems: The Rotating Gamma Systems (RGS) such as the GammaART-6000 are very similar to the Gamma Knife. Like the Gamma Knife, the RGS systems have a hemispherical collimator assembly with apertures directed to a common focal spot at the center of the hemisphere. The major difference is that instead of 192 static radioactive Cobalt-60 sources, the RGS systems have ˜30 sources that orbit the patient about the z-axis. The coordinate system reference is shown in FIG. 1. By rotating the sources about the z-axis, the total accumulated solid angle of beam trajectories is greater than that of the Gamma Knife; however, it is still limited to less than 2 steradian coverage. It must be noted that the radiation beams have a single orbital DOF about the z-axis.

Gantry-based Linear Accelerators & Particle Beam Systems: Gantry-based Linear Accelerators and Particle Beam Systems have a gantry that orbits a single radiation source around the z-axis. A gantry based linear accelerator and particle beam system is shown in FIG. 3. They have a patient bed that can rotate the patient about the y-axis. These systems rely on the same general technique: a collimated x-ray or particle beam is directed toward the stereotactically defined target volume and the gantry rotates the beam around the patient producing an arc of radiation focused on the target. A single arc is planar, and because it is oriented perpendicular to the z-axis, the entrance dose and exit dose trajectories overlap if the gantry is rotated more than 180 degrees. Then the patient couch is rotated about the y-axis and another arc is performed. In this manner, multiple non-coplanar arcs of radiation are delivered to the target volume.

Other Linear Accelerator Systems: Other Linear Accelerator systems, such as CyberKnife and Zap-X do not use a gantry to position the treatment head and radiation beams. CyberKnife is a compact linear accelerator treatment head mounted to a 6-DOF serial robotic arm. Zap-X utilizes a 2-DOF gimbal system to position its compact linear accelerator around the patient. These systems do not offer arc therapy; instead, they deliver many different non-coplanar beam trajectories one at a time in a step-and-shoot method. Although it is possible to deliver the beams from many angles with these systems, in clinical settings the total coverage is typically less than 2 steradians due to the exceptionally long treatment times. FIGS. 4 and 5 how this multiple static beam delivery technique might look for a few shots and many shots:

Beam Orientation: The orientation of the beam with respect to the patient is important. In the Gantry-based LINAC systems and all Proton/Particle Beam systems, the radiation beam is perpendicular to the z-axis of the patient. In the Gamma Knife and Rotating Gamma Systems, none of the beams are perpendicular to the z-axis of the patient. In CyberKnife (Linac), and Zap-x (Linac) systems the beams are not restricted to being perpendicular to the z-axis of the patient.

Coplanar vs Non-coplanar beam delivery techniques: Coplanar beam delivery is the delivery of multiple radiation beams where each beam trajectory lies in the same plane. An example of coplanar beam delivery is shown in FIG. 6 which depicts how a continuous radiation beam that is perpendicular to the z-axis is rotated about this axis 360 degrees Forming a single planar “arc” around the patient.

In FIG. 6 Step 1 shows the initial instantaneous single beam entering the skull and targeting an intracranial tumor. Step 2 shows how the instantaneous radiation beams start to form an arc as the gantry continuously orbits the patient 90 degrees about the z-axis. The accumulated beam entry angles are shown in light grey and the instantaneous beam is shown in dark gray. Steps 3, 4 & 5A show the accumulated and instantaneous beams after 180, 270, and 360-degree rotation about the z-axis. Step 5B shows the same as Step 5A but from a different view. As you can see in Step 5B all beam trajectories lie in the same plane and are therefore coplanar. Notice that after 180 degrees, the entrance and exit beams start to overlap with each other. This occurs because the beam is perpendicular to the z-axis about which it is rotated.

The first type of Non-coplanar arc therapy techniques, which are called Volumetric Modulated Arc Therapy (VMAT) and Intensity Modulated Arc Therapy (IMAT), utilize multiple, continuously moving sweeping arcs, each of which is oriented in a unique plane. In all arcs, the beam is oriented perpendicular to the axis of rotation, such that the entrance and exit beams share a common plane. FIG. 7 shows a depiction of how 3 sweeping arcs are non-coplanar in both an isometric view and a top-down view of the patient. It must be noted that only one arc is delivered at a time.

This type of Non-coplanar beam delivery greatly improves healthy tissue sparring by further increasing the accumulated solid angle of beam trajectories because it spreads out the dose delivered outside of the target volume through a greater volume of surrounding tissues. It is generally used by all modern radiosurgery and radiation therapy systems.

The second type of non-coplanar beam delivery technique is utilized by the RGS systems and is made possible because the orientation of the beams are not perpendicular to the z-axis about which they are rotated. In the RGS systems, the sources are pitched about the x-axis at various fixed angles toward the skull of the patient. Therefore, when the sources are orbited about the z-axis of the patient the entrance and exit beams no longer overlap in the same plane. This is depicted with a single source in FIG. 8.

Notice how the entrance and exit beams do not overlap and that even with a single rotation, the beam trajectories are non-coplanar. If you compare this figure with FIG. 6 above, you can see that the radiation dose outside of the target volume would be spread out through a larger volume and reduces the damage to the surrounding tissue structures. In the RGS systems, there are multiple sources orbiting the patient simultaneously. Therefore, with a single orbital rotation, multiple non-coplanar arcs are delivered simultaneously. FIG. 9 depicts a simplified version of this when multiple sources are pitched about the x-axis towards the patient's skull, as in the RGS systems.

### SUMMARY OF THE INVENTION

The subject radiosurgery system has a 7 DOF robotic gantry to deliver 22 simultaneous pencil beams of Cobalt-60-based Gamma Radiation, which all converge to a common focal spot called the radiation isocenter. An onboard hexapod robot connects the radiation treatment head to the rotating ring of the Gantry. The hexapod can move the radiation treatment head with 6 DOF (x, y, z, roll, pitch, and yaw) and the rotating ring can orbit the hexapod and treatment head, providing the 7th degree of freedom. The hexapod can move the treatment head with a fixed control point-meaning that the radiation isocenter position remains stationary while the radiation beams enter the patient through different trajectories. It can also move the radiation isocenter to a different position than the geometric isocenter. In either mode, the subject system gantry can rotate either continuously or dynamically (i.e. non-constant speeds or in either direction).

### BRIEF DESCRIPTION OF THE DRAWINGS

FIG. 1 is an illustration of the coordinate system referenced in the specification.

FIG. 2 is an illustration of a Gamma knife system.

FIG. 3 is an illustration of a gantry based linear accelerator and particle beam system.

FIG. 4 is an illustration of a multiple static beam delivery.

FIG. 5 is another illustration of a multiple static beam delivery.

FIG. 6 is an illustration of a coplanar beam delivery.

FIG. 7 is an illustration of three non-coplanar beam arcs.

FIG. 8 is an illustration of a second non-coplanar beam delivery.

FIG. 9 is an illustration of beam delivering involving multiple simultaneous arcs.

FIG. 10 is an illustration of the system of the subject invention.

FIG. 11 is an illustration of the hexapod component of the subject invention.

FIG. 12 is an illustration of the single beam trajectory of the subject invention.

FIG. 13 is an illustration of the multiple beam trajector's of the subject invention.

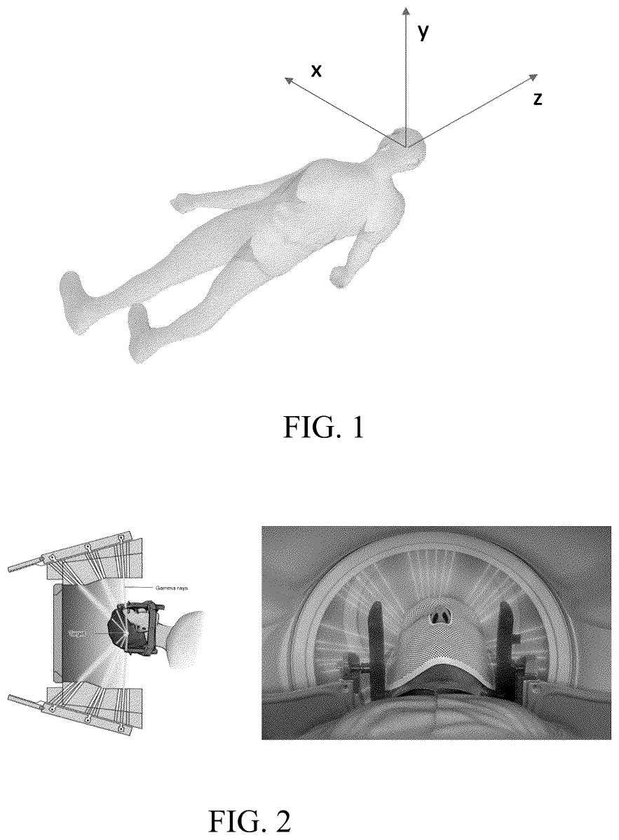

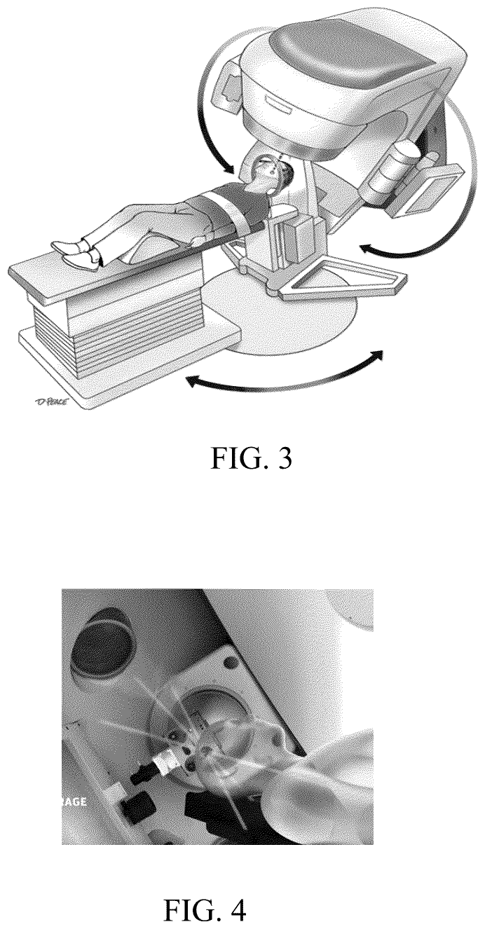

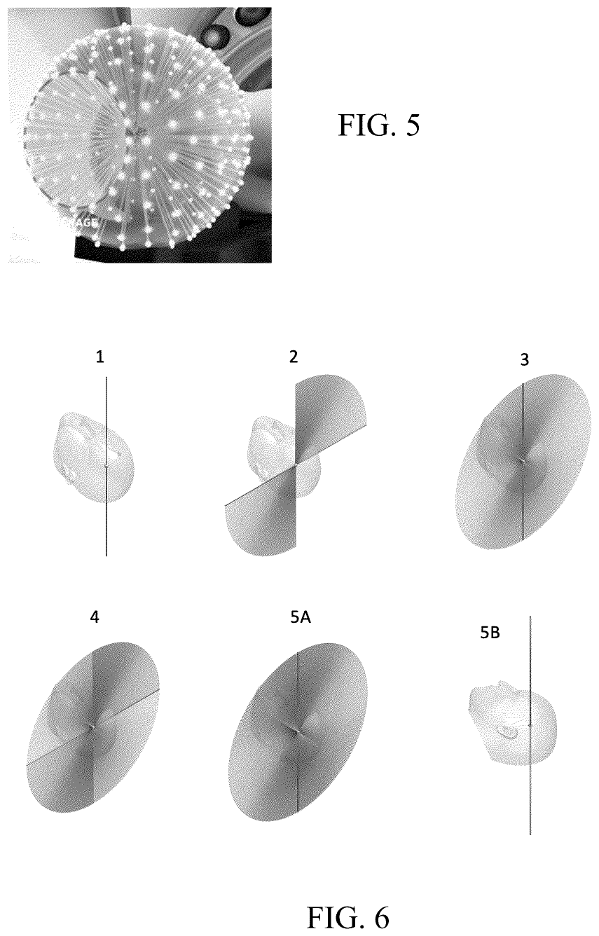

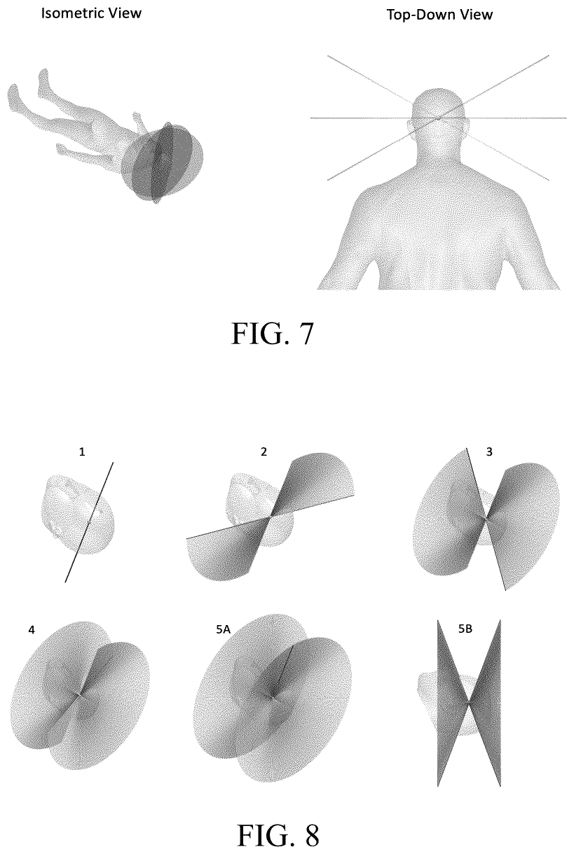

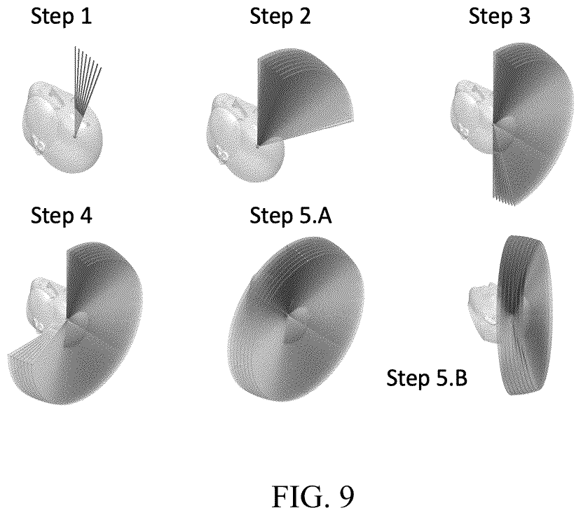

### DETAILED DESCRIPTION OF THE PREFERRED EMBODIMENTS

The subject invention Radiosurgery System's Beam Delivery Platform and Radiation Treatment Head are described in detail herein.

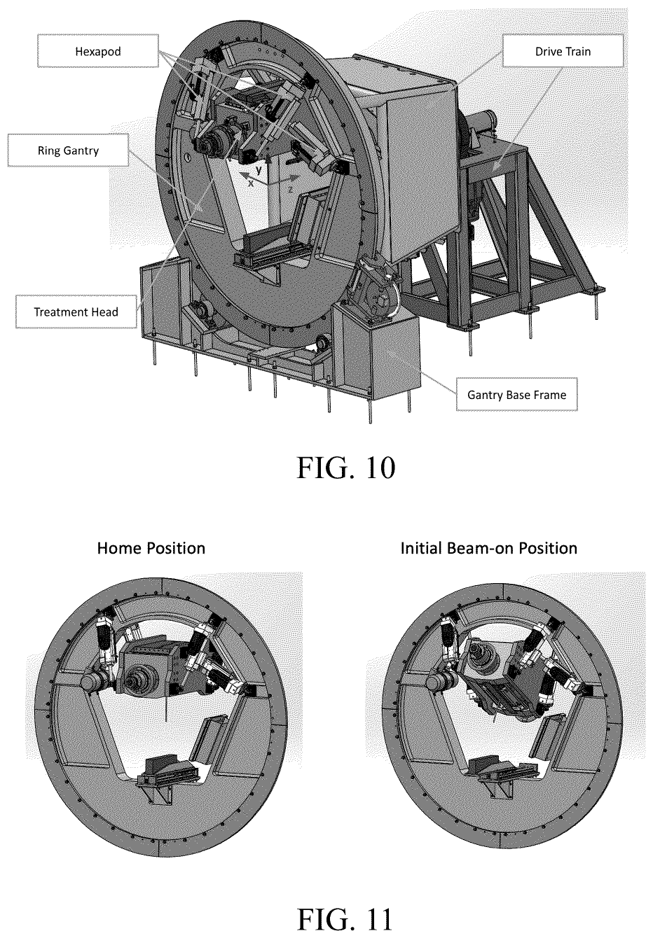

The subject system can thus deliver the radiation beams with 7 DOF dynamic motion in either isocentric, non-isocentric, or a combination of both techniques. The 22 simultaneous beams each exist within a unique non-coplanar arc as the rotating gantry orbits around the patient, and by dynamically moving the radiation head with the hexapod, the 22 noncoplanar beams are also dynamically changing, thus creating infinite non-coplanar arcs of radiation during the treatment procedure. This dynamic radiation delivery technique enables the subject system to provide the largest accumulated solid angle of beam delivery trajectories, therefore providing the best-in-class healthy tissue-sparing capabilities. The subject invention is shown in FIG. 10. In a preferred embodiment a six degree of freedom hexapod is connected to a rotating ring gantry and supports a treatment head connecting said treatment head to the ring gantry. As shown, ring gantry is supported on and rotates upon a gantry base frame. A drive train is provided. Those skilled in the art will understand that the hexapod system can be replaced with a six degrees of freedom parallel robotic system. With the subject radiosurgery system, the radiation treatment head can be pitched about the x-axis towards the skull of the patient as with the RGSs prior to turning on the beams. This is achieved by the 6 DOF hexapod that couples the treatment head to the ring gantry as depicted in FIG. 11.

In this figure, a rod that represents a single radiation beam is depicted for simplicity. During beam-on, the gantry can continuously rotate the radiation beam about the z-axis while the hexapod continuously rotates the radiation beam about the x-axis from the skull towards the body of the patient while maintaining a constant isocenter. In this example, the resulting trajectory of the beam will have the following distribution.

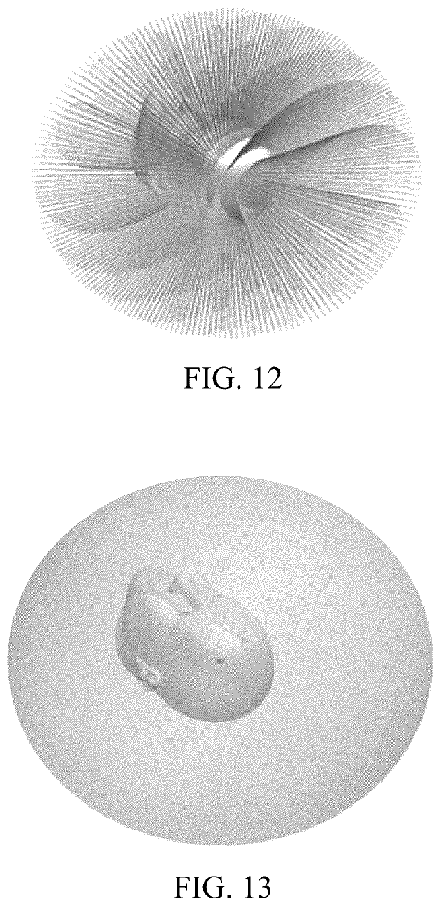

If you compare the single arc created with this simplified technique shown in FIG. 12 with those in FIGS. 6 and 8, you can see that the total accumulated solid angle of beam trajectories is significantly greater with this technique, which spreads the normal tissue dose throughout a much greater volume. In this simple example of the subject technique, we considered only a single radiation beam, that the orbital motion of the gantry and radiation beam about the z-axis was constant, and the rotational motion of the treatment head and beam about the x-axis was constant. If the same technique is applied with a radiation treatment head producing multiple beams, as with the current model of the subject radiosurgery system-which has 22 beams-then the total accumulated solid angle of beam trajectories will cover almost 4 steradians as shown in FIG. 13.

While the subject invention has been described in the context of delivering gamma beams those skilled in the art will understand that the treatment head can deliver proton beams, particle beams, electron beams and x-ray beams.

If the same technique is applied with fully dynamic motion capabilities of the subject gantry and hexapod subsystems, then infinite non-coplanar arcs of radiation can be delivered.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention. In addition, it should be understood that aspects of the various embodiments may be interchanged, both in whole, or in part. Therefore, the spirit, and scope of the invention should not be limited to the description of the preferred versions contained therein.

We claim:

1. A system for delivering converging radiation beams in the treatment of tumors comprising:
a hexapod system,
a radiation-head, and
a rotating gantry ring,
wherein the hexapod moves the radiation head with respect to the rotating gantry.

2. The system of claim 1 wherein:
the radiation head delivers an electron or x-ray beam.

3. The system of claim 1 wherein:
the radiation head delivers proton beams.

4. The system of claim 1 wherein:
the radiation head delivers Particle beams.

5. The system of claim 1 wherein:
the radiation head delivers Gamma beams.

6. A system for delivering converging radiation beams in the treatment of tumors comprising:
a six degree of freedom parallel robotic system,
a radiation-head, and
a rotating gantry ring,
wherein the robotic system moves the radiation head with respect to the rotating gantry.

7. The system of claim 6 wherein:
the radiation head delivers an electron or x-ray beam.

8. The system of claim 6 wherein:
the radiation head delivers proton beams.

9. The system of claim 6 wherein:
the radiation head delivers Particle beams.

10. The system of claim 6 wherein:
the radiation head delivers Gamma beams.

11. The system of claim 1 wherein:
the hexapod and rotating gantry provide for seven degrees of freedom for the radiation head.

12. The system of claim 6 wherein:
the parallel robotic systems and the rotating gantry provide for seven degrees of freedom for the radiation head.

13. The system of claim 1 wherein:
the radiation head can deliver radiation beams either isocentrically or non-isocentrically.

14. The system of claim 6 wherein:
the radiation head can deliver radiation beams either isocentrically or non-isocentrically.

15. The system of claim 1 wherein:
the radiation head can deliver up to 22 simultaneous radiation beams.

16. The system of claim 1 wherein:
the radiation head can deliver up to 22 simultaneous radiation beams.
